(19) United States
(12) Patent Application Publication
(10) Pub. No.: US 2022/0277206 A1
(43) Pub. Date: Sep. 1, 2022

(54) A DYNAMICALLY DISTORTED TIME WARPING DISTANCE MEASURE BETWEEN CONTINUOUS BOUNDED DISCRETE-TIME SERIES

(71) Applicant: OTSUKA PHARMACEUTICAL CO., LTD., Tokyo (JP)

(72) Inventors: Jonathan Roland Knights, Rockville, MD (US); Zahra Heidary, Rockville, MD (US)

(21) Appl. No.: 17/619,934

(22) PCT Filed: Jun. 17, 2020
(86) PCT No.: PCT/JP2020/024684

§ 371 Date: Dec. 16, 2021

Related U.S. Application Data
(60) Provisional application No. 62/862,644, filed on Jun. 17, 2019.

(51) Int. Cl. G06N 5/04; G16H 20/10

(52) U.S. Cl. CPC G06N 5/04; G16H 20/10

(57) ABSTRACT

Methods, systems, and computer programs for predicting an entity's adherence or non-adherence to a regimen. A method includes accessing observed attributes of an entity during a first time duration, accessing historical data describing attributes of another entity, where the historical data was previously obtained during respective second time durations, for each of the other entities, that are each equal in duration to the first time duration, filtering the historical data to only include historical data describing attributes of at least one other entity that satisfy a similarity threshold, the similarity threshold defining a relationship between the observed attributes and the historical data based on a distorted distance measure, collapsing the remaining historical data into one or more representative data, and for each representative data series, generating a similarity based prediction related to an outcome for the entity based on (i) the observed data and (ii) the representative data attributes.

### CROSS-REFERENCE TO RELATED APPLICATIONS

This application claims the benefit of U.S. Provisional Patent Application No. 62/862,644 filed Jun. 17, 2019, the entire contents of which is incorporated herein by reference.

### BACKGROUND ART

In conventional systems, a Euclidean distance can be used to determine a distance between respective data series.

### SUMMARY OF THE INVENTION

The present disclosure is directed towards a systems, methods, and computer programs for predicting a person's adherence or non-adherence to a regimen. The regimen may include a prescribed or non-prescribed regimen. The regimen can include a medicinal regimen, a therapeutic regimen, an exercise regimen, a treatment plan, or the like. Adherence or non-adherence to the regimen is predicted by comparing time series data that is indicative of a current person's level of adherence to a current regimen to time series data indicative of a representative patient of each of a plurality of different classes. This comparison is facilitated, inter alia, by using a novel similarity measure for relating sets of time series data. This novel similarity measure is based on a distorted distance between sets of time series. Use of this novel similarity measure can provide a complete picture of the similarity between different behavioral rhythmic, or cyclic time series in a manner that cannot be achieved using conventional distance measure such as Euclidean distance, which does not perform optimally for phase-shifted data series or data series having local patterns of dissimilarity.

According to one innovative aspect of the present disclosure, a data processing system for generating predictions using each of plurality of values in a data series is disclosed. The data processing system can include one or more processors and one or more storage devices storing instructions that, when executed by the one or more processors, cause the one or more processors to perform operations. In one aspect, the operations can include accessing a first data structure that stores observed data describing attributes of an entity during a first time duration, accessing a plurality of second data structures that each store historical data describing attributes of another entity, the historical data having been previously obtained during respective second time durations, for each of the other entities, that are each equal in duration to the first time duration, filtering the plurality of second data structures to only include those second data structures storing historical data describing attributes of at least one of the other entities that satisfy a similarity threshold, wherein the similarity threshold defines a relationship between the observed data and the historical data based on a distorted distance measure, collapsing the remaining second data structures into one or more representative data series that each include representative data attributes that are representative of the historical data describing attributes of one or more of the remaining second data structures, and for each the one or more representative data series, generating, by the data processing system, a similarity based prediction related to an outcome for the entity based on (i) the observed data and (ii) the representative data attributes in the representative data series.

Other versions include corresponding apparatus, methods, and computer programs to perform the actions of methods defined by instructions encoded on computer readable storage devices.

These and other versions may optionally include one or more of the following features. For instance, in some implementations, the similarity based prediction for the entity includes a prediction of how much treatment the entity will complete of a treatment plan.

In some implementations, one or more of the other entities are an entity that has completed a treatment plan, and one or more of the other entities are an entity that has not completed the treatment plan.

In some implementations, the second data structure that structures historical data describing attributes of other entities comprises a cloned portion of a historical database describing entity attributes.

In some implementations, the distorted distance is determined using dynamically distorted dynamic time warping.

In some implementations, the historical data describing attributes are of the same format as the observed data describing attributes of the entity.

In some implementations, the entity can be a human.

In some implementations, the operations can further include determining, based on the generated similarity based predictions, that the entity is most similar to one of the representative data series corresponding to a group of entities that did not complete a treatment plan, and generating notification data that, when processed by a user device, generates an alert message that prompts the entity to continue adherence to the treatment plan.

These and other aspects of the present disclosure are discussed in more detail in the detailed description below with reference to the accompanying drawings.

### BRIEF DESCRIPTION OF THE DRAWINGS

FIG. 1 is a contextual diagram of an example of a system for predicting patient adherence using a dynamically distorted time warping distance measure.

FIG. 2 is a flowchart of an example of a process for predicting patient adherence using a dynamically distorted time warming distance measure.

FIG. 3 is a block diagram of system having system components that can be used to generate representative entity data structures for predicting entity participation in a treatment plan.

### DESCRIPTION OF EMBODIMENTS

The present disclosure is directed towards systems, methods, and computer programs for predicting an entity's adherence or non-adherence to a regimen. The regimen may include a prescribed or non-prescribed regimen. The regimen can include a medicinal regimen, a therapeutic regimen, an exercise regimen, or the like. Adherence or non-adherence to the regimen is predicted by comparing time series data that is indicative of a current person's level of adherence to a current regimen to time series data indicative of a representative patient of each of a plurality of different classes. This comparison is facilitated, inter alia, by using a novel similarity measure for relating sets of time series data. This novel similarity measure is based on a distorted distance between sets of time series. An entity can include, for example, any living organism. The living organism can include a human person or a non-human animal.

By way of example, the regimen can include a system that requires a patch to be coupled to an entity's skin. In such instances, the patch can be configured to cause a substance such as a drug to be absorbed via skin of the entity over time when the patch is coupled to the skin. In such an implementation, adherence to the regimen is a function of the duration and consistency with which the entity wears the patch. Accordingly, predictions as to the person's adherence to the therapeutic regimen can include a prediction as to whether the user will continue wearing the patch or discontinue wearing the patch. However, the present disclosure need not be limited to such implementations. By way of another example, a system can use a patch that was coupled to the entity's skin in, for example, the entity's torso or stomach area that is configured to detect ingestions of a medication by the entity. The patch can detect ingestions of the medication based on data output by a sensor in the stomach of a person which has been embedded into a drug that was ingested by the person.

In such systems, identifying entities that are similar to each other with respect to data quality can include generation and evaluation of a similarity measure of data distance between a set of data representing each of the respective entities. The data representing each of the respective entities can include, for example, time series data. In some implementations, the similarity measure can properly account for local properties in the sets of data such as different durations of use, and periods of intermittent data. Such local properties are naturally occurring in implementations such as an entity wearing a patch that detects occurrences of medication or other substances ingested by a user as current usage data can only be detected and generated when an entity is wearing the patch.

The similarity measure of the present disclosure can also be used to ensure that regardless of which series were being compared to each other, each element of the measure would maintain its ability to define ‘similarity’ at a relatively similar level. By way of example, the distance distortion techniques described herein can be used to account for fluctuations in the local structure of the data such as time series data so that if respective data patterns were identical but slightly shifted on an axis such as a time axis, the shift along the axis would be accounted for and a system using aspects of the present disclosure would recognize the respective time series being the same, or otherwise matching, patterns.

This similarity measure is used in clustering items of data (that each have at least a threshold amount of similarity to each other). The clustered data items are then collapsed into a representative data series with a plurality of values. Each of these values is then used in generating a prediction of an outcome. In particular, the system accesses (e.g., receives) immediate, observed data (e.g., for an entity—such as a patient) and historical data (for other entities or patients, for example). The system collapses the historical data into one or more representative data series that each have a plurality of representative data attributes. For each of the representative data series, the system uses the representative data attributes in generating a prediction of an outcome for an entity represented by (or identified by) the immediate, observed data.

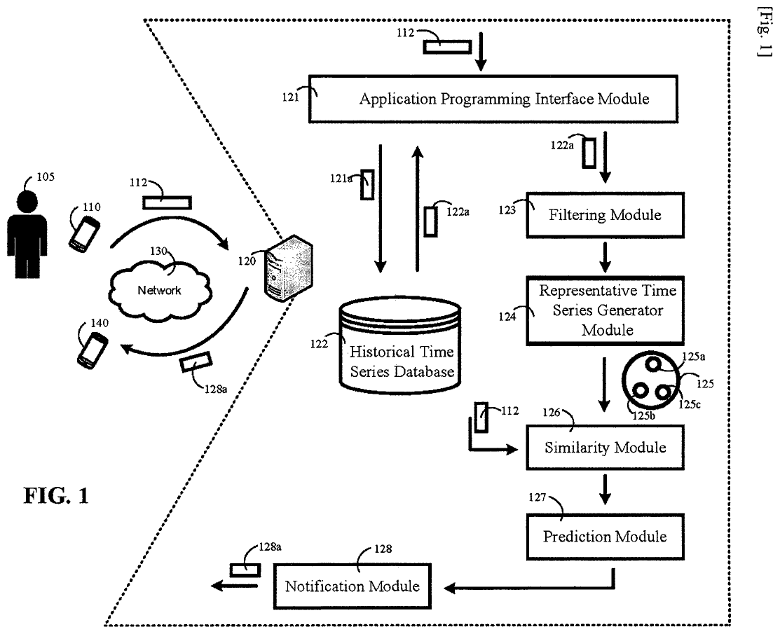

FIG. 1 is a contextual diagram of an example of a system 100 for predicting a patient's or entity's adherence to a regimen (or adherence to system detecting medicine or substance usage) using a dynamically distorted time warping distance measure. The system 100 can include a first user device 110, an application server 120, a network 130, and a second user device 140. The application server 120 can include application programming interface module 121, a historical time series data database 122, a filtering module 123, a representative time series data generator module 124, a similarity module 126, a prediction module 127, and a notification module 128.

In the example of FIG. 1, an entity such as a person 105 has begun a regimen such as a medicinal regimen. For example, the person 105 can begin taking a prescribed medicine. A first user device 110 can be used to collect data describing the person's 105 participation in the regimen and transmit the collected data series 112 describing the person's 105 participate in the regimen to the application server 120 via the network 130. The network 130 can include a wired Ethernet network, an optical network, a WiFi network, a LAN, a WAN, a cellular network, the Internet, or any combination thereof.

The user device 110 is depicted as a smartphone for sake of the illustration. And, in some implementations, the user device 110 can be a smartphone. For example, a smartphone can collect data describing the person's 105 participation in a regimen in a number of ways such as by syncing within one or more wearable devices that broadcast data describing the person's 105 participation in the regimen using shortwave radio signals such as Bluetooth. Then, the smartphone can transmit the collected data describing the person's 105 participate in the regimen to the application server 120. However, the present disclosure is not limited to a user device 110 that is a smartphone.

For example, in some implementations, the user device 110 can be any wearable device such as smartwatch, a patch that adheres to the person's 105 skin, a form of clothing having internet of things (IOT) sensors, or the like. In such implementations, the user device 110 can be capable of obtaining data describing the person's 105 participate in the regimen and transmitting the data describing the person's 105 participate in the regimen to the application server 120 without first transmitting the data describing the person's 105 participation in the regimen to another user device.

The collected data series 112 can be described as a current set of observed data describing the person's 105 participation in the regimen. The collected data series 112 can include multiple dimensions such as an adherence attribute and a time associated with the adherence attribute. In some implementations, the collected data series 112 can be data collected over a duration of time, with each adherence attribute being indexed by a particular time in the duration of time to create time series data. The duration of time can be periodic such as transmit collected data series 112 every predetermined number of minutes, every a predetermined number of hours, daily, weekly, or the like. Alternatively, the duration of time can be a non-periodic duration of time such as transmit collected data series 112 when the user device 110 has as network connection with the application server 120. The adherence attribute can include any attribute that relates to the person's 105 participation in the regimen such dose taken, missed dose, action performed, patch worn, patch not worn, or the like.

However, the present disclosure need not be so limited. For example, an adherence attribute can be any data generated by any engagement with any component of the medicinal or substance monitoring system during regimen monitoring. For example, an adherence attribute can be based on data generated by wearables, sensors such as electrodermal activity. Other examples of adherence attributes can include contextual data such as patient-reported data, passive data collection such as geolocation data, bluetooth data, mobile sensor data, or the like. In addition, other forms of adherence data can include data generated by other non-worn sensors such as smart-home sensors.

The application server 120 can include a number of processing modules that can be used to perform operations described herein. The processing modules can include an application programming interface module 121, a filtering module 123, a representative time series generator 124, a similarity module 126, a prediction module 127, and a notification module 128. For purposes of the present disclosure, a module can include one or more software instructions, one or more hardware components, or a combination thereof. Though the application server 120 is depicted as a single computer, the application server 120 can alternatively be implemented using multiple computers. In such instances, the functionality of the processing modules described herein may be implemented across one or more of the multiple computers. The application server 120 can also include, or be in communication with, a historical time series database 122 that stores historical time series data 122. Accordingly, the historical time series database 122 may be hosted by the application server 120 or remote from the application server 120.

The application server 120 can receive the collected data series 112 using an application programming interface module (API) 121. The API 121 an interface between the user device 110 or user device 140 and the application server 120. For example, the API can receive collected data such as collected data series 112 from different user devices such as user devices 110 of respective different entities. In addition, the API 121 can function to provide the notifications to the user device 110 or to another user device 140 after using the processing modules of the application server 120 to execute a process such as the process 200. The application server 120 can process collected data series 112, generate queries such as search query 121 used to search the historical time series data database 122, execute the search queries such as search query 121a, obtain search results 122a responsive to executed searches, and generate inputs 122a to the filtering module 123.

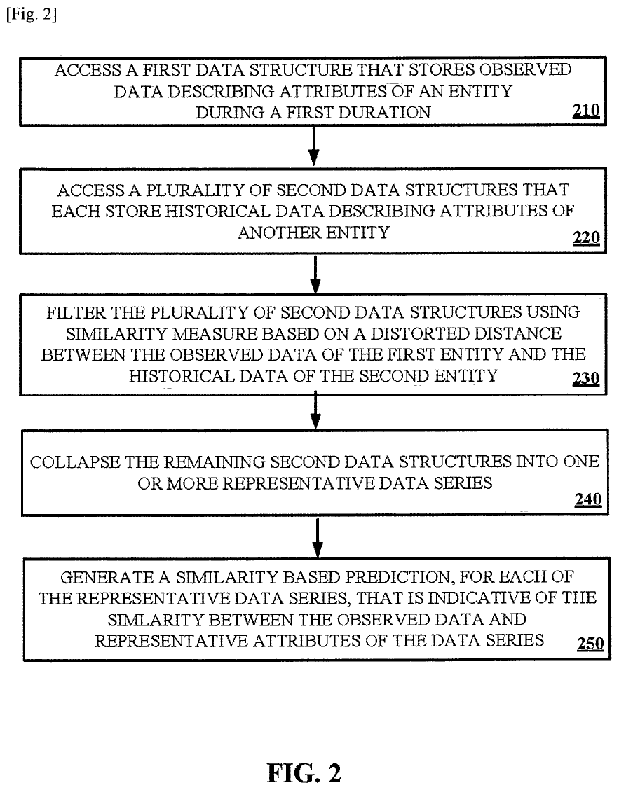

The historical time series database 122 can store collected data records aggregated by the application server 120. For example, entities such as person 105 using the system can have data describing their participation in a medical regimen be collected, and provided, to the application server 120 over time for use as an input to the application server 120 execution of one or more processes such as execution of the process 200 of FIG. 2. As this data is received by the application server 120, this data can be stored in the historical time series database 122. This data can include, for example, adherence attributes for a particular entity for a particular duration of time. The adherence attributes of the data can be indexed based on the time the data was generated, obtained, stored, or other indexing scheme. In some implementations, however, the adherence attributes can be indexed based on the time of engagement in a particular regimen by an entity such as person 105. For example, in some implementations, an adherence attribute can include data describing whether an entity wore a patch that is configured to detect ingestion of a medicine or substance or whether an entity did wear a patch that is configured to detect ingestion of a medicine or substance. In such instances, data stored in the historical time series database 122 can record that each include data indicating whether the entity corresponding to the record used or did not use the patch and the time that corresponds to each instance of use or non-use of the patch.

With reference to the example of FIG. 1, the API 121 can receive the collected data series 112. The API 121 can determine a duration of time associated with the collected data 121. The API 121 can generate a query 121a that includes, as a parameter of the query 121a, data describing an adherence attribute of the collected data 121, data describing a duration of time associated with the adherence attribute of the collected data 121, or both. The API 121 can execute the query 121a against the historical time series data 122 and obtain a set of search results 122a that include one or more records that each relate to an adherence attribute specified by the query 121a that is collected over a same duration of time as the duration specified by the query 121a. The search results 122a can be returned to the API 121, or otherwise obtained by the API 121. The API 121 can provide the set of search results 122a as an input to the filtering module 123.

The filtering module 123 can obtain the set of search results 122a as an input and identify a subset of the search results 122a for use in generating representative time series. Filtering can be achieved by evaluating historical time series in the set of search results 122a in view of the novel similarity measure of the present disclosure. This similarity measure is a distorted distance measure that accounts for peculiarities in a particular time series such as intermittent participate in a regimen in order to normalize a distance between uses that, once normalized, are of the same duration. In some implementations, the filtering module 123 can receive the input 112 and use the input 112 to filter the search results 122a.

The distorted distance measure is also fairly flexible with respect to minor fluctuations in the local structure of the data such that if patterns were identical but slightly shifted, these would be accounted for. The distorted distance measure can also account for complexities that come along with analyzing data that is inherently bounded on a narrow range—such as data representing a time-series of bounded data on the [0, 1] range. The measure is applicable for data sets and problems that determine a relative measure of similarity from one entity time series to a set of other entity time series in manner that is not a pure distance metric and needs further considerations in order to be leveraged downstream. In some implementations, the distorted distance measure is based on dynamic time warping.

In more detail, assume a given a set of discrete-time series

X={X₁, X₂, . . . Xₙ} where

Xᵢ={(tᵢ₁, xᵢ₁), (tᵢ₂, xᵢ₂), . . . , (tᵢₘ, xᵢₘ)}: x_{i*} ∈ [0, 1]

and

(tₓ_{ᵢⱼ}−tₓ_{ᵢ₍ⱼ₊₁₎}=c: ∀_{j∈[1,|X}_{ᵢ}_{|-1]})

define a reference length (duration) r_{D}=argmax(U_{X}|Xᵢ|). Where |Xᵢ| is the cardinality of each set in X. However, the present disclosure is not limited to equally spaced time points.

Given a set Xᵢ ∈ X, a measure of distance, D_{X}_{ᵢ}_{, X}_{_{j≠i}}∈ [1, n], can be defined that may simultaneously penalizes series of different lengths and periods of missing data (in one implementation defined as xᵢ=0) while still allowing similar patterns to occur at different time periods. This example describes distortion of, e.g., “periods of missing data,” however, the present disclose need not be so limited. Moreover, the present disclosure does not require that all dimensions of adherence data need be distorted. Instead, use of the present disclosure can include determination of a distortion strategy that can identify and select one or more adherence attributes that should be distorted in an effort to relate historical entities and the current entities with reference to the distorted adherence attribute. In some implementations, the particular adherence attribute distorted may be application specific.

With respect to one particular use case, series that are shorter than r_{D} may be penalized or padded. In such use cases, each series can be expanded to the reference length with X_{i(m→r}_{_{D}}₎=−v so that all series end up ultimately being of equal length. In some implementations, v=2 in order to heavily penalize early termination of data generation with respect to the reference duration. In theory, v could be any value in [1, ∞] but practically, for some implementations when the data is confined in [0, 1], does not need to be larger than 2.

The base for the distorted distance measure can be built on dynamic time warping, where the cumulative ‘distance’, D_{X}_{ᵢ}_{, X}_{_{j≠i}} between two series is recursively calculated as:

$${D}_{{X}_{i} , {X}_{j \neq i}} ( m ) = d ( {x}_{im} , {x}_{jm} ) + min \{ \begin{matrix}D ( {x}_{im - 1} , {x}_{jm} ) \\ D ( {x}_{im} , {x}_{jm - 1} ) \\ D ( {x}_{im - 1} , {x}_{jm - 1} )\end{matrix} \} : 1 \leq m , n \leq {r}_{D}$$

where d(xᵢₘ, xⱼₘ) is the point distance between (tᵢₘ, xᵢₘ) and (tⱼₘ, xⱼₘ). The ‘distance’ value is returned as D_{X}_{ᵢ}_{, X}_{_{j≠i}}(r_{d}).

In some implementations, to calculate the point-wise distance d(xᵢₘ, xⱼₘ), define:

$$d ( {x}_{im} , {x}_{jm} ) = \{ \begin{matrix}‖ {w}_{{x}_{ij , m}} * {x}_{im} ‖ , & {x}_{jm} = 0 , {x}_{im} > 0 \\ ‖ {w}_{{x}_{ij , m}} * {x}_{jm} ‖ , & {x}_{im} = 0 , {x}_{jm} > 0 \\ ‖ {x}_{im} , {x}_{jm} ‖ , & otherwise\end{matrix}$$

where ∥xᵢₘ, xⱼₘ∥ is the Euclidean distance between the two points (or vectors if in multivariate space) and Wₓ_{_{ij,m}} is a point and series specific distortion weight applied when one series is being compared to a missing value in another series. The double bracket notation is maintained so as to generalize to multivariate and non-equally spaced time values. The weighting term between {xᵢₘ ∈ Xᵢ, xⱼₘ ∈ Xⱼ} is operationally defined as:

$${w}_{{x}_{ij , m}} = ( v + 1 ) * \frac{1}{{f}_{i , j}} * argmax ( 1 , {s}_{im} , {s}_{jm} ) \\ {f}_{i , j} = \frac{argmin ( \vert {X}_{i} \vert , \vert {X}_{j} \vert )}{{r}_{D}}$$

In such implementations, the first term (v+1) represents the desire to maintain the same maximum point distance d(1, −v) as (xᵢ, 0: xᵢ>0). As each series may have different lengths relative to the reference, the influence of the within-series distance measure decreases as the series get shorter. To accommodate this distortion, f_{i,j} is incorporated to balance the influence of potentially different amounts of missing data relative to the reference on the distance measure so that the within-series distances maintain the same degree of influence on the overall distance between two series, regardless of length. Note that in the case both series are of the same length as the reference duration, f_{i,j}=1 and there is no need for distortion of the weight. In the example described here, the third term in the weight, S_{**}, represents a penalty for consecutive observations of missing data being compared to non-missing data across series (within the time frame argmax(|Xᵢ|, |Xⱼ|)). The logic for tracking the s_{**} terms is as follows:

sᵢₘ=0, sⱼₘ=0

for m in argmax(|Xᵢ|, |Xⱼ|):

if xᵢₘ=0 and xⱼₘ>0: sᵢₘ=sᵢ₍ₘ₋₁₎+1

else: sᵢₘ=0

if xⱼₘ=0 and xᵢₘ>0: sⱼₘ=sⱼ₍ₘ₋₁₎+1

else: sⱼₘ=0

This formulation describes a shorthand characterization, which does not account for edge cases of when m=0 (or 1 dependent on the indexes). For the case of m=0, for example, there would be no m−1 for use as a comparison.

The logic for not combining to a single weighting term is due to the fact that there may be asynchronous (alternating) periods of missing data across the two series and we are intentionally maintaining distinction between these swapping periods and single periods of consecutive missing data comparisons. The final methodology provides a framework for defining a generalizable pairwise distance measure between two discrete-time series of variable length in a set whose elements are proportions in [0, 1]. However, this should not be viewed as limited, as the elements can also be scaled to proportions having other ranges. For any given time series in the set, this distance measure is meant to identify the ‘k’ most similar series in the set. The final distance measure leverages the ability of dynamic time warping to find the minimum distance path between two time-series, while providing custom adaptations to the point-wise distance metric to penalize patterns of missing data that do not align with the comparator series or other patterns of data that are of interest.

The filtering module 123 can use the distorted distances measure described above to determine the relationship between each historical time series in the set of search results 122a and select the historical time series that are within a threshold distance of the collected data series 112. This subset of historical time series can be provided as an input to the representative time series generator 124.

The representative time series generator module 124 can obtain and process the subset of historical time series filtered from the set of search results 122a to generate representative time series 125. Each representative time series can represent a class of entities, with each class of entities corresponding to a different level of entity adherence to the regimen. Generation of the representative time series can include clustering the subset of filtered time series based on the distorted distance measure. Such clustering can be performed using clustering algorithms such as K-means clustering based on the distorted distance measure. However, the present disclosure is not limited to use of K-means clustering. Instead, in some implementations, other forms of clustering such as K-nearest neighbor clustering or hierarchical clustering may be used. Once clustered, the clusters of time series data can be collapsed into respective representative time series 125a, 125b, 125c that each include representative data attributes that are representative of the historical data describing attributes of one or more historical time series. Though some implementations product multiple different representative time series such as representative time series 125a, 125b, 125c, other implementations may only have a single representative time series. Such a single representative time series can be generated by collapsing a set of known historical time series without first performing clustering.

By way of example, each respective representative time series 125a, 125b, 125c can correspond to a representative entity that each had different levels of adherence to the regimen. For example, the representative time series 125a may represent adherence attributes of an entity that stopped participating in a regimen after a first time period (e.g., 2 days), the representative time series 125b may represent adherence attributes of an entity that stopped participating in a regimen after a second time period (e.g., 4 weeks), and the representative time series 125c may represent adherence attributes of an entity that continued participating in a regimen through completion of the regimen (e.g., 2 months). These representative time series and their respective time periods are merely examples, and in no way should be viewed as limiting. Instead, the present disclosure should be broadly interpreted such that the different representative time series 125a, 125b, 125c each representative of a historical entity with different levels of adherence to the regimen. The generated set of representative time series 125 can be provided as an input to the similarity module 126.

The similarity module 126 can receive the generated set of representative time series 125 as an input. The similarity module 126 can also obtain the collected data series 112 as another input. Then, the similarity module 126 can determine a level of similarity between the collected data series 112 and each representative time series 125a, 125b, 125c in the generated set of representative time series 125. The similarity between the collected data series 112 and each of the respective time series can be achieved based on evaluation of a distance measure between the collected data series 112 and each of the representative time series 125a, 125b, 125c in the generated set of representative time series 125. The distance measure can include the distorted distance measure of the present disclosure. The similarity module 126 can provide a plurality of values to the prediction module, where each value in the plurality of values is indicative of a level of similarity of the collected data series 112 to a particular representative time series 125a, 125b, 125c.

The prediction module 127 can evaluate each value of the plurality of values received from the similarity module 126. Based on this evaluation, the prediction module 127 can make a prediction as to the particular representative entity to which the current entity (i.e., person 105) corresponds. In some implementations, this can be achieved by selecting the representative time series that is related to the current entity's 105 current data series 112 by a shortest distorted distance. In some implementations, this may be achieved by determining the representative time series that is related to the current time series by the smallest similarity value. The prediction module 127 can provide data to a notification module 128 that indicates the predicted representative entity to which the current data series 112 corresponds.

The notification module 128 can generate notification data 128a, that when processed by a user device such as a smartphone of the user 105 or a user device 140 can be used to generate an alert notification to a user related to the classification of the current data series 112 classification. In some implementations, the generated notification data 128 can be used to encourage entity adherence to the regimen. For example, the prediction module can determine that the person 105 is most similar to a representative time series for a patient that stops participating in a regimen before the regimen is complete. In such instances, the generated notification data 128 can be sent to a smartphone of the user, a smart watch of the user, or user device 140 via the network 130 in order to cause the smartphone, smart watch, or user device 140 to generate alert notifications that encourage the entity 105 to continue the regimen, restart the regimen, or a combination thereof. In some implementations, the user device 140 can be the person's 105 smartphone that is separate from a user device 110 that is a wearable device such as a patch that detect consumption of a medicine or other substance. In other implementations, the user device 140 can be a user device of another user such as doctor that prescribed the regimen.

FIG. 2 is a flowchart of an example of a process 200 for predicting patient adherence using a dynamically distorted time warming distance measure. In general, the process 200 can include accessing a first data structure that stores observed data describing attributes of an entity during a first duration (210), accessing a plurality of second data structures that each store historical data describing attributes of another entity (220), filtering the plurality of second data structures using a similarity measure based on a distorted time distances between the observed data of the first entity and the historical data of the second entity (230), collapsing the remaining second data structures into one or more representative data series (240), and generating a similarity based prediction, for each of the representative data series, that is indicative of the similarity between the observed data and representative attributes of the data series (250).

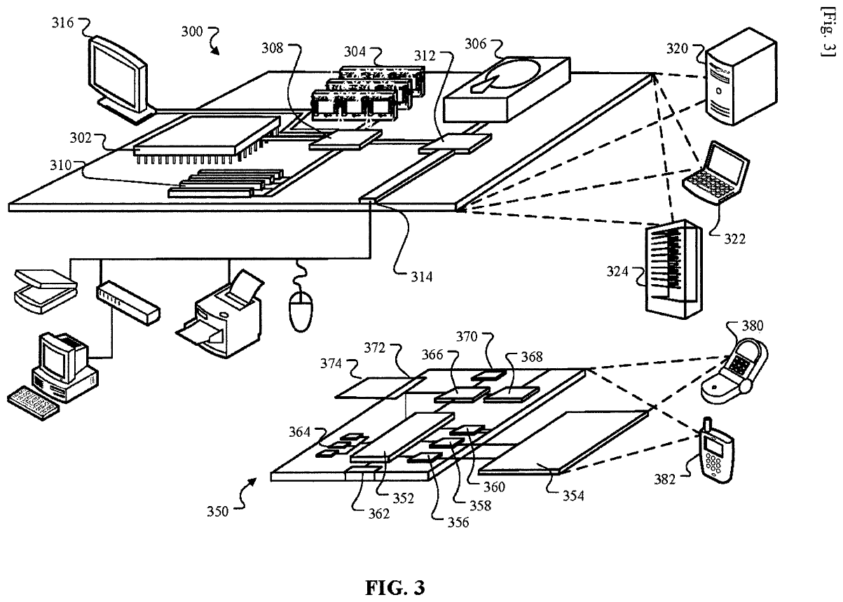

FIG. 3 is a block diagram of system 300 having system components that can be used to generate representative entity data structures for predicting entity participation in a treatment plan.

Computing device 300 is intended to represent various forms of digital computers, such as laptops, desktops, workstations, personal digital assistants, servers, blade servers, mainframes, and other appropriate computers. Computing device 350 is intended to represent various forms of mobile devices, such as personal digital assistants, cellular telephones, smartphones, and other similar computing devices. Additionally, computing device 300 or 350 can include Universal Serial Bus (USB) flash drives. The USB flash drives can store operating systems and other applications. The USB flash drives can include input/output components, such as a wireless transmitter or USB connector that can be inserted into a USB port of another computing device. The components shown here, their connections and relationships, and their functions, are meant to be exemplary only, and are not meant to limit implementations of the inventions described and/or claimed in this document.

Computing device 300 includes a processor 302, a memory 304, a storage device 306, a high-speed controller or interface 308 connecting to memory 304 and high-speed expansion ports 310, and a low speed interface 312 connecting to low speed bus 314 and storage device 306. Each of the components 302, 304, 306, 308, 310, and 312, are interconnected using various busses, and can be mounted on a common motherboard or in other manners as appropriate. The processor 302 can process instructions for execution within the computing device 300, including instructions stored in the memory 304 or on the storage device 306 to display graphical information for a GUI on an external input/output device, such as display 316 coupled to high speed controller or interface 308. In other implementations, multiple processors and/or multiple buses can be used, as appropriate, along with multiple memories and types of memory. Also, multiple computing devices 300 can be connected, with each device providing portions of the necessary operations, e.g., as a server bank, a group of blade servers, or a multi-processor system.

The memory 304 stores information within the computing device 300. In one implementation, the memory 304 is a volatile memory unit or units. In another implementation, the memory 304 is a non-volatile memory unit or units. The memory 304 can also be another form of computer-readable medium, such as a magnetic or optical disk.

The storage device 306 is capable of providing mass storage for the computing device 300. In one implementation, the storage device 306 can be or contain a computer-readable medium, such as a floppy disk device, a hard disk device, an optical disk device, or a tape device, a flash memory or other similar solid state memory device, or an array of devices, including devices in a storage area network or other configurations. A computer program product can be tangibly embodied in an information carrier. The computer program product can also contain instructions that, when executed, perform one or more methods, such as those described above. The information carrier is a computer- or machine-readable medium, such as the memory 304, the storage device 306, or memory on processor 302.

The high speed controller 308 manages bandwidth-intensive operations for the computing device 300, while the low speed controller 312 manages lower bandwidth intensive operations. Such allocation of functions is exemplary only. In one implementation, the high-speed controller or interface 308 is coupled to memory 304, display 316, e.g., through a graphics processor or accelerator, and to high-speed expansion ports 310, which can accept various expansion cards (not shown). In the implementation, low-speed controller 312 is coupled to storage device 306 and low-speed expansion port 314. The low-speed expansion port, which can include various communication ports, e.g., USB, Bluetooth, Ethernet, wireless Ethernet can be coupled to one or more input/output devices, such as a keyboard, a pointing device, microphone/speaker pair, a scanner, or a networking device such as a switch or router, e.g., through a network adapter. The computing device 300 can be implemented in a number of different forms, as shown in the figure. For example, it can be implemented as a standard server 320, or multiple times in a group of such servers. It can also be implemented as part of a rack server system 324. In addition, it can be implemented in a personal computer such as a laptop computer 322. Alternatively, components from computing device 300 can be combined with other components in a mobile device (not shown), such as device 350. Each of such devices can contain one or more of computing device 300, 350, and an entire system can be made up of multiple computing devices 300, 350 communicating with each other.

The computing device 300 can be implemented in a number of different forms, as shown in the figure. For example, it can be implemented as a standard server 320, or multiple times in a group of such servers. It can also be implemented as part of a rack server system 324. In addition, it can be implemented in a personal computer such as a laptop computer 322. Alternatively, components from computing device 300 can be combined with other components in a mobile device (not shown), such as device 350. Each of such devices can contain one or more of computing device 300, 350, and an entire system can be made up of multiple computing devices 300, 350 communicating with each other.

Computing device 350 includes a processor 352, memory 364, and an input/output device such as a display 354, a communication interface 366, and a transceiver 368, among other components. The device 350 can also be provided with a storage device, such as a micro-drive or other device, to provide additional storage. Each of the components 350, 352, 364, 354, 366, and 368, are interconnected using various buses, and several of the components can be mounted on a common motherboard or in other manners as appropriate.

The processor 352 can execute instructions within the computing device 350, including instructions stored in the memory 364. The processor can be implemented as a chipset of chips that include separate and multiple analog and digital processors. Additionally, the processor can be implemented using any of a number of architectures. For example, the processor 310 can be a CISC (Complex Instruction Set Computers) processor, a RISC (Reduced Instruction Set Computer) processor, or a MISC (Minimal Instruction Set Computer) processor. The processor can provide, for example, for coordination of the other components of the device 350, such as control of user interfaces, applications run by device 350, and wireless communication by device 350.

Processor 352 can communicate with a user through control interface 358 and display interface 356 coupled to a display 354. The display 354 can be, for example, a TFT (Thin-Film-Transistor Liquid Crystal Display) display or an OLED (Organic Light Emitting Diode) display, or other appropriate display technology. The display interface 356 can comprise appropriate circuitry for driving the display 354 to present graphical and other information to a user. The control interface 358 can receive commands from a user and convert them for submission to the processor 352. In addition, an external interface 362 can be provide in communication with processor 352, so as to enable near area communication of device 350 with other devices. External interface 362 can provide, for example, for wired communication in some implementations, or for wireless communication in other implementations, and multiple interfaces can also be used.

The memory 364 stores information within the computing device 350. The memory 364 can be implemented as one or more of a computer-readable medium or media, a volatile memory unit or units, or a non-volatile memory unit or units. Expansion memory 374 can also be provided and connected to device 350 through expansion interface 372, which can include, for example, a SIMM (Single In Line Memory Module) card interface. Such expansion memory 374 can provide extra storage space for device 350, or can also store applications or other information for device 350. Specifically, expansion memory 374 can include instructions to carry out or supplement the processes described above, and can include secure information also. Thus, for example, expansion memory 374 can be provide as a security module for device 350, and can be programmed with instructions that permit secure use of device 350. In addition, secure applications can be provided via the SIMM cards, along with additional information, such as placing identifying information on the SIMM card in a non-hackable manner.

The memory can include, for example, flash memory and/or NVRAM memory, as discussed below. In one implementation, a computer program product is tangibly embodied in an information carrier. The computer program product contains instructions that, when executed, perform one or more methods, such as those described above. The information carrier is a computer- or machine-readable medium, such as the memory 364, expansion memory 374, or memory on processor 352 that can be received, for example, over transceiver 368 or external interface 362.

Device 350 can communicate wirelessly through communication interface 366, which can include digital signal processing circuitry where necessary. Communication interface 366 can provide for communications under various modes or protocols, such as GSM voice calls, SMS, EMS, or MMS messaging, CDMA, TDMA, PDC, WCDMA, CDMA2000, or GPRS, among others. Such communication can occur, for example, through radio-frequency transceiver 368. In addition, short-range communication can occur, such as using a Bluetooth, Wi-Fi, or other such transceiver (not shown). In addition, GPS (Global Positioning System) receiver module 370 can provide additional navigation- and location-related wireless data to device 350, which can be used as appropriate by applications running on device 350.

Device 350 can also communicate audibly using audio codec 360, which can receive spoken information from a user and convert it to usable digital information. Audio codec 360 can likewise generate audible sound for a user, such as through a speaker, e.g., in a handset of device 350. Such sound can include sound from voice telephone calls, can include recorded sound, e.g., voice messages, music files, etc. and can also include sound generated by applications operating on device 350.

The computing device 350 can be implemented in a number of different forms, as shown in the figure. For example, it can be implemented as a cellular telephone 380. It can also be implemented as part of a smartphone 382, personal digital assistant, or other similar mobile device.

Various implementations of the systems and methods described here can be realized in digital electronic circuitry, integrated circuitry, specially designed ASICs (application specific integrated circuits), computer hardware, firmware, software, and/or combinations of such implementations. These various implementations can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms “machine-readable medium” “computer-readable medium” refers to any computer program product, apparatus and/or device, e.g., magnetic discs, optical disks, memory, Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor.

To provide for interaction with a user, the systems and techniques described here can be implemented on a computer having a display device, e.g., a CRT (cathode ray tube) or LCD (liquid crystal display) monitor for displaying information to the user and a keyboard and a pointing device, e.g., a mouse or a trackball by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

The systems and techniques described here can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the systems and techniques described here, or any combination of such back end, middleware, or front end components. The components of the system can be interconnected by any form or medium of digital data communication, e.g., a communication network. Examples of communication networks include a local area network (“LAN”), a wide area network (“WAN”), and the Internet.

The computing system can include clients and servers. A client and server are generally remote from each other and typically interact through a communication network. The relationship of client and server arises by virtue of computer programs running on the respective computers and having a client-server relationship to each other.

### OTHER EMBODIMENTS

A number of embodiments have been described. Nevertheless, it will be understood that various modifications can be made without departing from the spirit and scope of the invention. In addition, the logic flows depicted in the figures do not require the particular order shown, or sequential order, to achieve desirable results. In addition, other steps can be provided, or steps can be eliminated, from the described flows, and other components can be added to, or removed from, the described systems. Accordingly, other embodiments are within the scope of the following claims.

1. A data processing system for generating predictions with each value in a data series, the data processing system comprising one or more processors and one or more storage devices storing instructions that, when executed by the one or more processors, cause the one or more processors to perform operations, the operations comprising:
accessing a first data structure that stores observed data describing attributes of an entity during a first time duration;
accessing a plurality of second data structures that each store historical data describing attributes of another entity, the historical data having been previously obtained during respective second time durations, for each of the other entities, that are each equal in duration to the first time duration;
filtering the plurality of second data structures to only include those second data structures storing historical data describing attributes of at least one of the other entities that satisfy a similarity threshold, wherein the similarity threshold defines a relationship between the observed data and the historical data based on a distorted distance measure;
collapsing the remaining second data structures into one or more representative data series that each include representative data attributes that are representative of the historical data describing attributes of one or more of the remaining second data structures; and
for each the one or more representative data series, generating, by the data processing system, a similarity based prediction related to an outcome for the entity based on (i) the observed data and (ii) the representative data attributes in the representative data series.

2. The data processing system of claim 1,
wherein the similarity based prediction for the entity includes a prediction of how much treatment the entity will complete of a treatment plan.

3. The data processing system of claim 1,
wherein one or more of the other entities are an entity that has completed a treatment plan, and
wherein one or more of the other entities are an entity that has not completed the treatment plan.

4. The data processing system of claim 1, wherein the second data structure that structures historical data describing attributes of other entities comprises a cloned portion of a historical database describing entity attributes.

5. The data processing system of claim 1, wherein the distorted distance is determined using dynamically distorted dynamic time warping.

6. The data processing system of claim 1, wherein the historical data describing attributes are of the same format as the observed data describing attributes of the entity.

7. The data processing system of claim 1, wherein the entity includes a human.

8. The data processing system of claim 1, the operations further comprising:
determining, based on the generated similarity based predictions, that the entity is most similar to one of the representative data series corresponding to a group of entities that did not complete a treatment plan; and
generating notification data that, when processed by a user device, generates an alert message that prompts the entity to continue adherence to the treatment plan.

9. A method comprising:
accessing a first data structure that stores observed data describing attributes of an entity during a first time duration;
accessing a plurality of second data structures that each store historical data describing attributes of another entity, the historical data having been previously obtained during respective second time durations, for each of the other entities, that are each equal in duration to the first time duration;
filtering the plurality of second data structures to only include those second data structures storing historical data describing attributes of at least one of the other entities that satisfy a similarity threshold, wherein the similarity threshold defines a relationship between the observed data and the historical data based on a distorted distance measure;
collapsing the remaining second data structures into one or more representative data series that each include representative data attributes that are representative of the historical data describing attributes of one or more of the remaining second data structures; and
for each the one or more representative data series, generating, by the data processing system, a similarity based prediction related to an outcome for the entity based on (i) the observed data and (ii) the representative data attributes in the representative data series.

10. The method of claim 9,
wherein the similarity based prediction for the entity includes a prediction of how much treatment the entity will complete of a treatment plan.

11. The method of claim 9,
wherein one or more of the other entities are an entity that has completed a treatment plan, and
wherein one or more of the other entities are an entity that has not completed the treatment plan.

12. The method of claim 9, wherein the second data structure that structures historical data describing attributes of other entities comprises a cloned portion of a historical database describing entity attributes.

13. The method of claim 9, wherein the distorted distance is determined using dynamically distorted dynamic time warping.

14. The method of claim 9, wherein the historical data describing attributes are of the same format as the observed data describing attributes of the entity.

15. The method of claim 9, wherein the entity includes a human.

16. The method of claim 9, the method further comprising:
determining, based on the generated similarity based predictions, that the entity is most similar to one of the representative data series corresponding to a group of entities that did not complete a treatment plan; and
generating notification data that, when processed by a user device, generates an alert message that prompts the entity to continue adherence to the treatment plan.

17. A non-transitory computer-readable medium storing software comprising instructions executable by one or more computers which, upon such execution, cause the one or more computers to perform operations comprising:
accessing a first data structure that stores observed data describing attributes of an entity during a first time duration;
accessing a plurality of second data structures that each store historical data describing attributes of another entity, the historical data having been previously obtained during respective second time durations, for each of the other entities, that are each equal in duration to the first time duration;
filtering the plurality of second data structures to only include those second data structures storing historical data describing attributes of at least one of the other entities that satisfy a similarity threshold, wherein the similarity threshold defines a relationship between the observed data and the historical data based on a distorted distance measure;
collapsing the remaining second data structures into one or more representative data series that each include representative data attributes that are representative of the historical data describing attributes of one or more of the remaining second data structures; and
for each the one or more representative data series, generating, by the data processing system, a similarity based prediction related to an outcome for the entity based on (i) the observed data and (ii) the representative data attributes in the representative data series.

18. The computer-readable medium of claim 17,
wherein the similarity based prediction for the entity includes a prediction of how much treatment the entity will complete of a treatment plan.

19. The computer-readable medium of claim 17,
wherein one or more of the other entities are an entity that has completed a treatment plan, and
wherein one or more of the other entities are an entity that has not completed the treatment plan.

20. The computer-readable medium of claim 17, wherein the second data structure that structures historical data describing attributes of other entities comprises a cloned portion of a historical database describing entity attributes.

21. The computer-readable medium of claim 17, wherein the distorted distance is determined using dynamically distorted dynamic time warping.

22. The computer-readable medium of claim 17, wherein the historical data describing attributes are of the same format as the observed data describing attributes of the entity.

23. The computer-readable medium of claim 17, wherein the entity includes a human.

24. The computer-readable medium of claim 17, the operations further comprising:
determining, based on the generated similarity based predictions, that the entity is most similar to one of the representative data series corresponding to a group of entities that did not complete a treatment plan; and
generating notification data that, when processed by a user device, generates an alert message that prompts the entity to continue adherence to the treatment plan.
